(19) United States
(12) Patent Application Publication
(10) Pub. No.: US 2008/0234778 A1
(43) Pub. Date: Sep. 25, 2008

(54) SEAL ELEMENT AND CONTACT SOCKET FOR MEDICAL IMPLANT

(76) Inventor: Ronald REBENTISCH, Berlin (DE)

Correspondence Address: Intellectual Property Department;DEWITT ROSS & STEVENS S.C., 6th Floor, 2 E. Mifflin St., Madison, WI, 53703, US

(21) Appl. No.: 12/031,069

(22) Filed: Feb. 14, 2008

(30) Foreign Application Priority Data
Mar. 24, 2007 (DE) 10 2007 014 217.1

(51) Int. Cl. A61N 1/00

(52) U.S. Cl. 607/36

(57) ABSTRACT

A contact socket for connecting an electrode line to an implantable medical device has a socket housing which has a plug receptacle having an elongate receiving chamber having a longitudinal axis for receiving a plug along the longitudinal axis, the plug receptacle being formed by a one-piece cast part, extending over the entire length of the plug receptacle, made of a permanently-elastic compound, in which electrical contact elements made of electrically conductive material are inserted.

### FIELD OF THE INVENTION

The present invention relates to a contact socket for connecting an electrode line to an implantable medical device, in particular to an implantable cardiac pacemaker or an implantable cardioverter/defibrillator.

### BACKGROUND OF THE INVENTION

Contact sockets of this type have a socket housing having a plug receptacle having an elongate receiving chamber for receiving a matching plug.

In cardiac pacemakers or defibrillators, the contact socket is used for connecting a plug at the proximal end of an electrode line to the housing of the cardiac pacemaker or defibrillator to form a seal and simultaneously to ensure a secure and reliable electrical connection. Typically, electrodes provided in the area of the distal end of the electrode line are electrically connected to electrical and/or electronic components in the interior of the housing of the cardiac pacemaker or defibrillator via the plug at the proximal end of the electrode line. These electrical and/or electronic components include, for example, stimulation pulse generators or input amplifiers for amplifying electrical potentials recorded in the heart.

The contact socket is typically situated in a terminal part, which itself comprises insulating plastic and is connected tightly to the remaining housing of a cardiac pacemaker or defibrillator. The remaining housing typically comprises metal and encloses the electrical and/or electronic components. The electrical connection between the components in the interior of the housing of the cardiac pacemaker and the terminal part as well as the contact socket situated in the terminal part occurs via electrical leadthroughs.

Electrically conductive contact elements are situated in the contact socket itself, through which corresponding counter contacts of the plug on the electrode line may be contacted, i.e., electrically connected thereto.

To allow replaceability of electrodes and/or cardiac pacemakers and defibrillators of various producers, the terminal dimensions of the plug on the electrode line and the corresponding dimensions of the contact socket in the terminal part of the cardiac pacemaker or defibrillator are standardized. The currently most widespread standard is typically referred to as IS-1. A newer, more current standard is known under the name IS-4.

### SUMMARY OF THE INVENTION

The present invention is based on the object of providing a contact socket which allows reliable production of a terminal part, in particular for a cardiac pacemaker or defibrillator, and which additionally ensures a reliable electrical connection to the counter contacts on the plug of an electrode line or which ensures a reliable seal of the contact socket having inserted electrode line plug in relation to surrounding media, such as blood.

This object is achieved according to the present invention by a contact socket for connection to an electrode line on an implantable medical device, which has a socket housing on a plug receptacle, in whose elongate receiving chamber a plug of an electrode line is to be inserted. The plug receptacle is a one-piece cast part, which extends over the entire length of the plug receptacle and is formed by a permanently-elastic compound. Electrical contact elements made of electrically conductive material are inserted into the plug receptacle.

The cast part is preferably an injection-molded part and the permanently-elastic compound preferably comprises silicone or at least contains silicone. Silicone has a suitably adjustable elasticity and makes the insertion of an electrode line plug into the receiving chamber easier because of good sliding properties.

A one-piece plug receptacle simplifies the production of a contact socket and/or a terminal housing for an implantable cardiac pacemaker or cardioverter/defibrillator and solely because of this improves the reliability of the device. In addition, a one-piece plug receptacle is advantageous in regard to the tightness of the contact socket.

The latter aspect comes to bear in particular if the receiving chamber enclosed by the plug receptacle is only open at one longitudinal end for inserting a plug and is closed at the other longitudinal end. An embodiment variant of this type is accordingly preferred.

The tightness of the contact socket is further improved according to a preferred embodiment variant in that the one-piece cast part already contains sealing ribs.

The sealing ribs are preferably situated directly adjacent to contact elements inserted in the plug receptacle. Receiving chambers are preferably provided for these contact elements in the plug receptacle, so that the one-piece cast part, viewed in the longitudinal direction, preferably alternatingly has receiving chambers for one contact element each and sealing ribs situated between them. All cross sections of the plug receptacle preferably have a circular basic shape. Through openings are preferably only provided in the area of the contact receiving chambers, which open outwardly starting from the particular contact receiving chamber in relation to the plug receptacle. Accordingly, the plug receptacle and the one-piece cast part do not have a circular internal cross-section in the area of these through openings.

The external cross-section of the one-piece cast part and/or the plug receptacle is preferably continuously circular over the entire length of the plug receptacle, so that the plug receptacle has the shape of a circular cylinder viewed from the outside.

The internal diameter of the cast part and the plug receptacle is less than 3.2 mm in the area of the sealing ribs and is otherwise greater than 3.2 mm.

In addition to three contact receiving chambers for contact elements for contacting ring contacts, a contact receiving chamber for receiving a terminal screw contact is preferably provided in the area of a closed end of the plug receptacle.

As already noted, a preferred material for the plug receptacle and/or the cast part forming the plug receptacle is silicone, preferably having a Shore hardness between 30 and 70, more preferably 50. The cast part forming the plug receptacle is additionally preferably temperature-treated to ensure a desired hardness.

In addition, the plug receptacle comprising elastic plastic is preferably enclosed by hard plastic. For example, the plug receptacle may be enclosed by epoxide resin, which preferably also defines the external contour of the terminal housing.

The present invention also relates to a method for producing the plug receptacle described above.

This method preferably comprises the following method steps:
providing an external casting mold and a casting core and assembling them, so that they define a free space, which determines both the internal and also the external contours of the plug receptacle,
filling the free space with liquid silicone,
causing the silicone to cure, which thus forms the plug receptacle,
removing the external casting mold after the plug receptacle is cured,
causing the plug receptacle to swell, and
removing the casting core when the plug receptacle is swollen.

By causing the plug receptacle to swell, it widens enough that the casting core may be removed without, for example, fine sealing ribs of the cast part forming the plug receptacle made of silicone being destroyed. Simultaneously, the electrical contact elements may be inserted easily into the particular contact receiving chambers when the plug receptacle is swollen.

A suitable agent for causing the plug receptacle to swell is n-heptane.

The plug receptacle is preferably temperature treated after the curing, either before it is caused to swell or even better after the insertion of the contact elements.

After the insertion of the contact elements, they may be contacted with electrical connection lines via through openings leading to the contact elements. Subsequently, the plug receptacle may be embedded using a hard plastic, preferably using epoxide resin.

The present invention will be explained in greater detail on the basis of an exemplary embodiment with reference to the figures.

### BRIEF DESCRIPTION OF THE DRAWINGS

In the figures:

FIG. 1: shows a schematic side view of a cardiac pacemaker;

FIG. 2: shows a side view of an electrode line plug;

FIG. 3: shows a longitudinal section through a contact socket of the cardiac pacemaker from FIG. 1; and

FIG. 4: shows a longitudinal section and a front view of a plug receptacle for a contact socket, similar to that from FIG. 3;

### DETAILED DESCRIPTION OF PREFERRED VERSIONS OF THE INVENTION

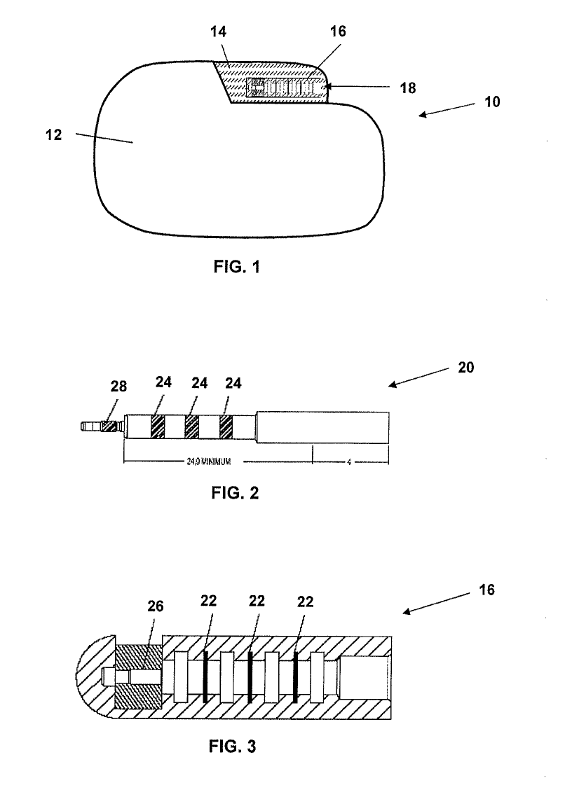

FIG. 1 shows a cardiac pacemaker 10 having a metal housing 12, which houses electrical and/or electronic components of the cardiac pacemaker, and a terminal housing 14 connected tightly to the metal housing 12. The terminal housing 14 houses a contact socket 16, which includes a receiving chamber 18 for receiving an electrode line plug 20, as is shown in FIG. 2. FIG. 3 is an enlarged view of the contact socket 16 and its receiving chamber 18 in longitudinal section.

The contact socket has three identical contact elements 22, which are used to contact annular counter contact faces 24 of the electrode line plug 20.

In addition, the contact socket 16 has a fourth contact element 26, which is used to receive a terminal screw contact 28 on the outer free end of the electrode line plug 20.

The essential component of the contact socket 16 is, besides the contact elements 22 and 26, a plug receptacle 30, which comprises a one-piece injection-molded part made of silicone.

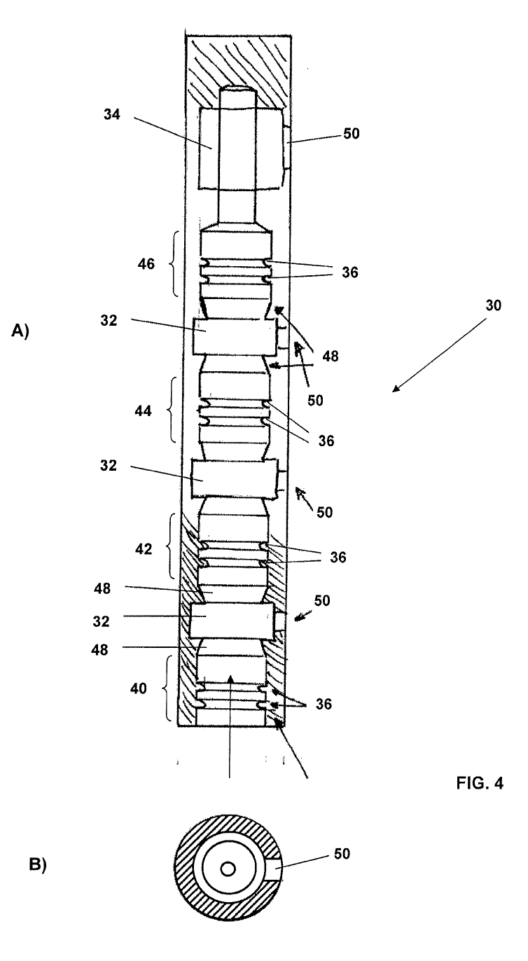

FIG. 4 shows a longitudinal section through the plug receptacle 30 in FIG. 4A and a front view of the open side of the plug receptacle 30 in FIG. 4B.

As may be inferred from FIG. 4A, the plug receptacle 30 has a total of four contact receiving chambers, of which three contact receiving chambers 32 are used for receiving contact elements for contacting ring contacts 24 of an electrode line plug, while the fourth contact receiving chamber 34 is used to receive a terminal screw contact.

With inserted contact elements, the plug receptacle 30 defines a receiving chamber 18, for receiving a plug like the plug 20 from FIG. 2. The receiving chamber 18 is closed at one end in proximity to the contact receiving chamber for the terminal screw contact and is open at the opposite end for inserting a plug.

Two sequential, annular seals 36 are provided between this opening at one longitudinal end of the receiving chamber 18 and the first contact receiving chamber 32, whose clearance is preferably less than 3.2 mm. Corresponding seals 36 are provided in pairs between the contact receiving chambers 32 and 34 as an integral component of the one-piece contact receptacle 30.

The seals 36 define a first seal area 40, a second seal area 42, a third seal area 44, and a fourth seal area 46.

The contact receiving chambers 32 are each delimited in the longitudinal direction by fixing elements 48 for the contact elements to be inserted. Starting from each of the contact receiving chambers 32 and/or 34, one through opening 50 extends up into the exterior of the plug receptacle 30 and allows the particular contact element to be contacted from the outside using an electric line after insertion into the plug receptacle 30.

The plug receptacle 30 is produced as a one-piece cast part made of silicone in the injection molding method. The internal chamber enclosed by the plug receptacle 30 is thus predefined by a casting core, which is removed after the injection molding and curing of the silicone in that the plug receptacle, having the casting core still initially inserted (but otherwise having the casting mold removed), is bathed in n-heptane, so that the silicone swells and the internal chamber of the plug receptacle 30 widens in such a way that the casting core may be removed without, for example, the fine, rib-like seals 36 being damaged. In this swollen state of the plug receptacle 30, the contact elements may also be inserted into the contact receiving chambers 32 and/or 34.

After the insertion of the contact elements, the plug receptacle 30 may shrink again, in that the n-heptane vaporizes. Subsequently, the entire plug receptacle 30 having inserted contact elements is temperature treated to increase the cross-linking of the already cured silicone and thus set a desired material hardness and elasticity.

The contact elements may then be contacted from the outside using electric lines and subsequently the plug receptacle having inserted and contacted contact elements may be embedded using epoxide resin, so that as a result the terminal housing 14 shown in FIG. 1 results.

The invention is not intended to be limited to the preferred versions of the invention described above, but rather is intended to be limited only by the claims set out below. Thus, the invention encompasses all different versions that fall literally or equivalently within the scope of these claims.

What is claimed is:

1. A contact socket for connecting an electrode line to an implantable medical device, the contact socket having a socket housing with a plug receptacle therein, the plug receptacle:
a. having an elongate receiving chamber with a longitudinal axis for receiving a plug along the longitudinal axis,
b. being formed by a one-piece cast part which extends over the entire length of the plug receptacle, the cast part being made of a permanently elastic compound,
c. having electrical contact elements made of electrically conductive material inserted within the cast part.

2. The contact socket according to claim 1 wherein the one-piece cast part has sealing ribs.

3. The contact socket according to claim 1 wherein the permanently elastic compound includes silicone.

4. The contact socket according to claim 3 wherein the plug receptacle includes silicone having a Shore hardness between 30 and 70, preferably 50, and is enclosed by a comparatively hard transparent plastic.

5. The contact socket according to claim 1 wherein the hard transparent plastic includes epoxide resin.

6. The contact socket according to claim 1 wherein the plug receptacle has a circular cylindrical exterior.

7. The contact socket according to claim 1 wherein the plug receptacle:
a. has an open diameter of more than 3.2 mm over the majority of its length, and
b. has sealing ribs extending inwardly from its diameter, with the open diameter being less than 3.2 mm within the sealing ribs.

8. The contact socket according to claim 1 wherein the plug receptacle has contact receiving chambers each extending over a portion of the total length of the receiving chamber, each contact receiving chamber having an enlarged internal diameter so that a particular contact receiving chamber may receive an electrical contact element made of electrically conductive material.

9. The contact socket according to claim 8 wherein the one-piece cast part has sealing ribs which are situated between the contact receiving chambers along the length of the plug receptacle.

10. The contact socket according to claim 8 wherein the plug receptacle has at least one through opening in the area of each of the contact receiving chambers, each through opening leading outwardly from one of the contact receiving chambers.

11. The contact socket according to claim 8 wherein the plug receptacle has four contact receiving chambers, three of which are implemented to receive electrical ring contacts, and a fourth of which is implemented to receive a terminal screw contact.

12. The contact socket according to claim 1 wherein the contact socket is part of a terminal part of a cardiac pacemaker and/or a cardioverter/defibrillator.

13. A contact socket for connecting an electrode line to an implantable medical device, the contact socket having a socket housing with a plug receptacle therein, the plug receptacle:
a. being integrally molded of elastic material;
b. having an elongated receiving chamber extending therein, the elongated receiving chamber opening onto a plug insertion aperture and including, along its length:
(1) two or more contact receptacle chambers, the contact receptacle chambers defining areas of enlarged inner diameter within the elongated receiving chamber;
(2) electrical contact elements made of electrically conductive material, each electrical contact element:
(a) having:
(i) an outer circumference,
(ii) an opposing inner circumference wherein a plug may be fit,
(iii) a first side extending between the outer circumference and the inner circumference, and
(iv) an opposing second side extending between the outer circumference and the inner circumference,
(b) being fit within one of the contact receptacle chambers, wherein the plug receptacle:
(i) surrounds at least a substantial portion of the outer circumference of the electrical contact element, and
(ii) bounds at least a portion of each of the first and second sides of the electrical contact element.

14. The contact socket of claim 13 wherein each electrical contact element cannot be removed from its contact receptacle chamber without either:
a. swelling the plug receptacle to an enlarged size, or
b. destructively opening the plug receptacle to access the contact receptacle chamber.

15. The contact socket of claim 13 wherein the elongated receiving chamber also includes one or more sealing ribs, each sealing rib:
a. defining an area of reduced inner diameter within the elongated receiving chamber, and
b. being situated prior to one or more of the contact receptacle chambers as the elongated receiving chamber extends inwardly from the plug insertion aperture.

16. The contact socket of claim 13 wherein at least one of the electrical contact elements has a threaded inner circumference.

17. The contact socket of claim 16 wherein the electrical contact element of the final contact receptacle chamber within the depth of the elongated receiving chamber has a threaded inner circumference.

18. The contact socket of claim 13 wherein each of the electrical contact elements includes an electric line leading therefrom, each electrical line extending through a respective through opening extending outwardly through the plug receptacle from one of the contact receptacle chambers.

19. A method for producing a plug receptacle for a socket housing of an implantable medical device, the method including the steps of:
a. providing an external casting mold about an internal casting core to define a free space therebetween, the free space determining the internal and external contours of the plug receptacle,
b. filling the free space with fluid elastic compound,
c. at least partially solidifying the elastic compound to form the plug receptacle,
d. removing the external casting mold,
e. causing the plug receptacle to swell, and
f. removing the casting core.

20. The method of claim 19 wherein the fluid elastic compound is injection molded into the free space.

21. The method of claim 19 wherein the step of causing the plug receptacle to swell includes the step of exposing the plug receptacle to n-heptane.

22. The method of claim 19 wherein one or more contact elements, each being formed of electrically conductive material, are inserted within the plug receptacle when it is in the swollen state.

23. The method of claim 22 wherein the plug receptacle is temperature treated after removal of the core to attain a different hardness.

24. The method of claim 22 wherein the contact elements are connected to electrical lines after the insertion within the plug receptacle.

25. The method of claim 24 wherein the plug receptacle is at least partially embedded within a comparatively hard plastic after insertion of the contact elements and connection to the electrical lines.
